(19) United States
(12) Patent Application Publication
(10) Pub. No.: US 2013/0178542 A1
(43) Pub. Date: Jul. 11, 2013

(54) MONOCATIONIC POLYHYDROXYL COMPOUNDS

(71) Applicants: Joseph Deavenport, Lake Jackson, TX (US); Nicole Brehm, Lake Jackson, TX (US)

(72) Inventors: Joseph Deavenport, Lake Jackson, TX (US); Nicole Brehm, Lake Jackson, TX (US)

(73) Assignee: DOW GLOBAL TECHNOLOGIES LLC, Midland, MI (US)

(21) Appl. No.: 13/822,077

(22) PCT Filed: Sep. 21, 2011
(86) PCT No.: PCT/US2011/052506

§ 371 Date: Mar. 11, 2013

Related U.S. Application Data
(60) Provisional application No. 61/388,226, filed on Sep. 30, 2010.

(51) Int. Cl. C07C 211/63

(52) U.S. Cl. CPC C07C 211/63
USPC 514/788; 564/292; 564/281

(57) ABSTRACT

Described are novel monocationic polyhydroxyl compounds and their uses in personal care compositions.

### FIELD

The present invention relates to novel monocationic polyhydroxyl compounds and their uses in personal care compositions.

### BACKGROUND

Polyhydroxyl compounds, or polyols, have a number of uses, from raw materials used in the manufacture of urethane foams to humectants for personal care products like shaving foams, lotions, and shampoos.

Quaternary ammonium compounds are also useful in a number of applications, such as for disinfectants, surfactants, fabric softeners, and conditioners in shampoos.

Despite the number of available conventional compounds, there is a strong need for novel compounds with properties to differentiate performance or offer synergistic effects in areas of interest, particularly in personal care compositions.

### DETAILED DESCRIPTION

In one embodiment, the present invention provides compounds, including salts, of the Formula (I):

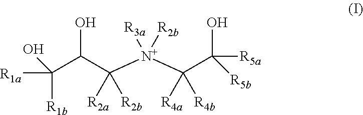

wherein:
R₁ₐ, R_{1b}, R₂ₐ, R_{2b}, R₄ₐ, R_{4b}, R₅ₐ, and R_{5b}, are, independently, H, optionally substituted C1-C6 alkyl, or R₄ₐ and R_{4b} may cooperate to form a cycloalkyl; and
R₃ₐ and R_{3b} are, independently, optionally substituted C1-C6 alkyl, with the proviso that:
   when R₁ₐ, R_{1b}, R₂ₐ, R_{2b}, R₄ₐ, R_{4b}, and R₅ₐ are each H, and R₃ₐ and R_{3b} are each —CH₃, then R_{5b} is other than —CH₂OH;
   when R₁ₐ, R_{1b}, R₂ₐ, R_{2b}, R₄ₐ, R_{4b}, and R₅ₐ are each H, and R₃ₐ and R_{3b} are each —CH₃, then R_{5b} is other than H;
   when R₁ₐ, R_{1b}, R₂ₐ, R_{2b}, R₄ₐ, R_{4b}, R₅ₐ, and R_{5b} are each H, and R₃ₐ and R_{3b} are each —CH₂CH(OH)CH₂OH, then R_{5b} is other than —CH₂OH; and
   when R₁ₐ, R_{1b}, R₂ₐ, R_{2b}, R₄ₐ, R_{4b}, R₅ₐ, and R_{5b} are each H, and R₃ₐ is —CH₃, then R_{3b} is other than —CH₂CH₂OH.

The term “optionally substituted” as used herein means that the groups in question are either unsubstituted or substituted with one or more groups, radicals or moieties, selected from halogen, hydroxy, amino or carboxy. When the groups in question are substituted with more than one substituent, the substituents may be the same or different. In one embodiment, the optional substituent is selected to produce a cosmetically acceptable compound. “Cosmetically acceptable” refers to ingredients typically used in personal care compositions, and is intended to underscore that materials that are toxic, irritating, or unpleasant smelling when present in the amounts typically found in personal care compositions are not contemplated as part of the present invention. In one embodiment, the optional substituent is one or more hydroxy groups.

“Alkyl” means a saturated monovalent linear or branched aliphatic hydrocarbon radical. Representative examples include, but are not limited to methyl, ethyl, propyl, isopropyl, butyl, isobutyl, tert-butyl, sec-butyl, pentyl, isopentyl, neopentyl, hexyl, isohexyl, and the like.

The term “cycloalkyl” denotes a saturated monocyclic or bicyclic cycloalkyl group. Examples of cycloalkyl groups include cyclopropyl, cyclobutyl, cyclopentyl, cyclohexyl, cycloheptyl, cyclooctyl, and the like. In one embodiment, the cycloalkyl is cyclohexyl or cyclopentyl.

Salts means that a counter-ion is present, preferably halogen, more preferably CT.

In one embodiment, R₁ₐ and R_{1b} are each H.

In one embodiment, R₂ₐ and R_{2b} are each H.

In one embodiment, R₃ₐ and R_{3b} are each —CH₃. In an alternative embodiment, R₃ₐ and R_{3b} are each —CH₂CH(OH)CH₂OH. In yet another alternative embodiment, R₃ₐ and R_{3b} are each —CH₂CH₂OH. In yet another alternative embodiment, R₃ₐ is —(CH₂)₃CH₃ and R_{3b} is —CH₂CH₂OH. In yet another alternative embodiment, R₃ₐ is —CH₃ and R_{3b} is —CH₂CH(OH)CH₂OH.

In one embodiment, R₄ₐ is —CH₂OH.

In one embodiment, R_{4b} is —CH₂CH₃.

In one embodiment, R₄ₐ and R_{4b} cooperate to form a cyclohexyl group.

In one embodiment, the present invention provides compounds, including salts, of the Formula (II):

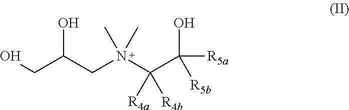

wherein:

R₄ₐ, R_{4b}, R₅ₐ, and R_{5b}, are, independently, H, optionally substituted C1-C6 alkyl, or R₄ₐ and R_{4b} may cooperate to form a cycloalkyl;

with the proviso that:
when R₄ₐ, R_{4b}, and R₅ₐ are each H, then R_{5b} is other than —CH₂OH; and
when R₄ₐ, R_{4b}, and R₅ₐ are each H, then R_{5b} is other than H.

Non-limiting examples of compounds of Formula II include the reaction products of 3-chloro-1,2-propanediol and 2-(Dimethylamino)-2-(hydroxymethyl)-1,3-chloro-1,2-propanediol and 2-(Dimethylamino)-2-ethyl-1,3-chloro-1,2-propanediol and 2-Dimethylamino-2-methylpropanol, 3-chloro-1,2-propanediol and 3-Dimethylamino-4-hydroxyoctane, 3-chloro-1,2-propanediol and Dimethylaminobutanol, 3-chloro-1,2-propanediol and 2-Dimethylamino-1,3-propanediol, 3-chloro-1,2-propanediol and 2-Dimethylamino-2-methyl-1,3-propanediol, 3-chloro-1,2-propanediol and 1-(Dimethylamino)cyclohexanemethanol, 3-chloro-1,2-propanediol and N,N-Dimethyl-2-amino-2-methylbutan-1-ol, 3-chloro-1,2-propanediol and 1-Dimethylamino-2-propanol, and 3-chloro-1,2-propanediol.

It is understood that many other embodiments where R₁ₐ and R_{1b} are not both methyl are contemplated, for example, the reaction products of 3-chloro-1,2-propanediol and N-Methyl Diethanolamine, 3-chloro-1,2-propanediol and Triisopropanolamine, 3-chloro-1,2-propanediol and 2-(Diethylamino)ethanol, 3-chloro-1,2-propanediol and monomethylamine, 3-chloro-1,2-propanediol and N-ethyldiethanol amine, 3-chloro-1,2-propanediol and 2-(Diisopropylamino)ethanol, 3-chloro-1,2-propanediol and N-Butyldiethanol amine, 3-chloro-1,2-propanediol and N-tert-Butyldiethanol amine, 3-chloro-1,2-propanediol and 2-(Dibutylamino)ethanol, and 3-chloro-1,2-propanediol and VANTEX®-T amine (available from TAMINCO N.V, Belgium).

In one embodiment, the present invention provides methods for providing humectancy in a personal care composition, comprising including the compound of Formula I into the personal care composition. The ingredients used, and their proportions and manner of addition, are familiar to those versed in conventional personal care compositions, including, optionally, cosmetically acceptable emollients, moisturizers, conditioners, oils, sunscreens, surfactants, emulsifiers, preservatives, rheology modifiers, colorants, preservatives, pH adjustors, propellants, reducing agents, fragrances, foaming or de-foaming agents, tanning agents, depilatory agents, astringents, antiseptics, deodorants, antiperspirants, insect repellants, bleaches, tighteners, anti-dandruff agents, adhesives, polishes, strengtheners, fillers, barrier materials, or biocides.

In one embodiment, the present invention provides hair care compositions containing the compound of Formula I.

In one embodiment, the present invention provides skin care compositions containing the compound of Formula I.

### EXAMPLES

The following examples are for illustrative purposes only and are not intended to limit the scope of the present invention.

### Example 1

### Reaction of 3-chloro-1,2-propanediol and dimethylaminobutanol to afford 2-(2,3-dihydroxypropyldimethylammonium)-1-butanol chloride

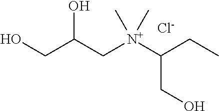

A 100 mL round bottom, jacketed, flask was equipped with a magnetic stirbar and placed on a stir-plate. The temperature was set to 20° C. and monitored with a thermocouple. Dimethylaminobutanol, 23.56 g (0.2 mol), was added to the reactor flask. Next, 21.86 g (0.2 mol) of 3-chloro-1,2-propanediol was added dropwise over about 10 minutes to prevent too exothermic a reaction, i.e., the temperature was maintained at less than 30° C. The mixture was allowed to react for about 1 hour at 20° C., and then increased to 40° C.

3 g of 49.6% NaOH solution was added to increase the pH to about 11. The mixture reacted for an additional 3 hours forming a thick white grey suspension. The temperature was increased to 50° C. and roughly 20 g of water was added to decrease the viscosity of the mixture. The mixture was then allowed to react for about 12 hours, NMR confirmed reaction completion. The pH was adjusted from about 12 to roughly neutral using concentrated HCl.

¹³C NMR spectra acquired from a Bruker 300 MHz spectrometer (samples prepared as ˜30 wt % in D₂O) confirmed the title compound: DEPT NMR (250 MHz, D2O) 10.5, (17.6, 17.7), (49.7, 50.3, 50.5), 56.7, 63.7, (65.4, 65.6, 66.1), 68.1, (76.1, 76.4).

### Example 2

### Reaction of 3-chloro-1,2-propanediol and 3-Dimethylamino-4-hydroxyoctane to afford 3-(2,3-dihydroxypropyldimethylammonium)-4-hydroxyoctane chloride

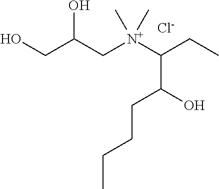 [SMILES: CCCCC(O)C(CC)[N+](C)(C)CC(O)CO ; [Cl-]]

The title compound is prepared substantially according to the protocol of Example 1, except that the conditions and amounts of reactants may vary, typically a 1:1 mol ratio with a slight excess of 3-chloro-1,2-propanediol, but which factors are well within the skill of one ordinarily skilled in the art.

### Example 3

### Reaction of 3-chloro-1,2-propanediol and 2-Dimethylamino-1,3-propanediol to afford 2-(2,3-dihydroxypropyldimethylammonium)-1,3-propanediol chloride

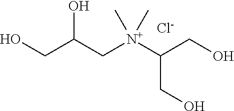

The title compound is prepared substantially according to the protocol of Example 1, except that the conditions and amounts of reactants may vary, typically a 1:1 mol ratio with a slight excess of 3-chloro-1,2-propanediol, but which factors are well within the skill of one ordinarily skilled in the art.

### Example 4

### Reaction of 3-chloro-1,2-propanediol and 2-Dimethylamino-2-methyl-1,3-propanediol to afford 2-(2,3-dihydroxypropyldimethylammonium)-2-methyl-1,3-propanediol chloride

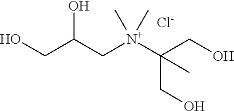

The title compound is prepared substantially according to the protocol of Example 1, except that the conditions and amounts of reactants may vary, typically a 1:1 mol ratio with a slight excess of 3-chloro-1,2-propanediol, but which factors are well within the skill of one ordinarily skilled in the art.

### Example 5

### Reaction of 3-chloro-1,2-propanediol and N,N-Dimethyl-2-amino-2-methylbutan-1-ol to afford 2-(2,3-dihydroxypropyldimethylammonium)-2-methyl-1-butanol chloride

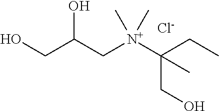

The title compound is prepared substantially according to the protocol of Example 1, except that the conditions and amounts of reactants may vary, typically a 1:1 mol ratio with a slight excess of 3-chloro-1,2-propanediol, but which factors are well within the skill of one ordinarily skilled in the art.

### Example 6

### Reaction of 3-chloro-1,2-propanediol and 2-(Dimethylamino)-2-ethyl-1,3-propanediol to afford 2-(2,3-dihydroxypropyldimethylammonium)-2-ethyl-1,3-propanediol chloride

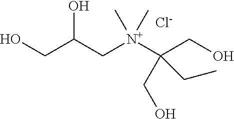

The title compound is prepared substantially according to the protocol of Example 1, except that the conditions and amounts of reactants may vary, typically a 1:1 mol ratio with a slight excess of 3-chloro-1,2-propanediol, but which factors are well within the skill of one ordinarily skilled in the art.

### Example 7

### Reaction of 3-chloro-1,2-propanediol and 2-(Dimethylamino)-2-(hydroxymethyl)-1,3-propanediol to afford 2-(2,3-dihydroxypropyldimethylammonium)-2-hydroxymethyl-1,3-propanediol chloride

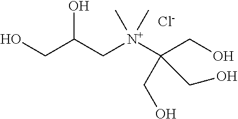

The title compound is prepared substantially according to the protocol of Example 1, except that the conditions and amounts of reactants may vary, typically a 1:1 mol ratio with a slight excess of 3-chloro-1,2-propanediol, but which factors are well within the skill of one ordinarily skilled in the art.

### Example 8

### Reaction of 3-chloro-1,2-propanediol and 2-Dimethylamino-2-methylpropanol to afford 2-(2,3-dihydroxypropyldimethylammonium)-2-methylpropanol

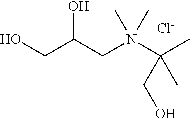

The title compound is prepared substantially according to the protocol of Example 1, except that the conditions and amounts of reactants may vary, typically a 1:1 mol ratio with a slight excess of 3-chloro-1,2-propanediol, but which factors are well within the skill of one ordinarily skilled in the art.

### Example 9

### Reaction of 3-chloro-1,2-propanediol and VANTEX®-T amine to afford 2,3-dihydroxypropyl(di)hydroxyethylbutylammonium chloride

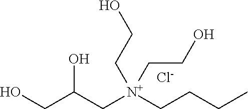 [SMILES: CCCC[N+](CCO)(CCO)CC(O)CO ; [Cl-]]

The title compound is prepared substantially according to the protocol of Example 1, except that the conditions and amounts of reactants may vary, typically a 1:1 mol ratio with a slight excess of 3-chloro-1,2-propanediol, but which factors are well within the skill of one ordinarily skilled in the art.

### Example 10

### Reaction of 3-chloro-1,2-propanediol and 1-(Dimethylamino)cyclohexanemethanol to afford 2,3-dihydroxypropyldimethylcyclohexanemethanolammonium chloride

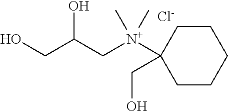 [SMILES: C[N+](C)(CC(O)CO)C1(CO)CCCCC1 ; [Cl-]]

The title compound is prepared substantially according to the protocol of Example 1, except that the conditions and amounts of reactants may vary, typically a 1:1 mol ratio with a slight excess of 3-chloro-1,2-propanediol, but which factors are well within the skill of one ordinarily skilled in the art.

### Example 11

### Reaction of 3-chloro-1,2-propanediol and 1-Dimethylamino-2-propanol to afford 3-(2,3-dihydroxypropyldimethylammonium)-2-propanol chloride

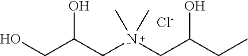

The title compound was prepared substantially according to the protocol of Example 1, except that 19.97 g (0.19 mol) of 1-Dimethylamino-2-propanol was reacted with 20.79 g (0.19 mol) of 3-chloro-1,2-propanediol; 0.53 g 49.6% NaOH solution was added to bring the pH above 12; the mixture was allowed to react for 5 hours at 20° C.; and then the pH was increased to 12.3 (using 0.48 g of 49.6% NaOH solution) and the temperature was increased to 40° C., and after about 1.5 hours, the temperature was increased to 60° C.

¹³C NMR spectra acquired from a Bruker 300 MHz spectrometer (samples prepared as ˜30 wt % in D₂O) confirmed the title compound: DEPT NMR (250 MHz, D2O) 21.3, (52.5, 52.6, 52.9), (62.1, 62.2), 63.7, (66.1, 66.2), (66.9, 67.3), (70.1, 70.5).

### Example 12

### Reaction of 3-chloro-1,2-propanediol and monomethylamine to afford tris(2,3-dihydroxypropyl)methylammonium chloride

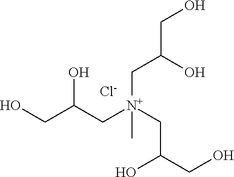 [SMILES: C[N+](CC(O)CO)(CC(O)CO)CC(O)CO ; [Cl-]]

The title compound was prepared substantially according to the protocol of Example 1, except that 9.24 moles 3-chloro-1,2-propanediol was reacted with 2.96 moles monomethylamine; sufficient 49.6% NaOH solution to achieve a pH of about 10.5 to about 11 was added; the reaction was maintained at 25° C. for an hour, then increased to about 50° C. for about 2.5 hours; additional 49.6% NaOH solution to achieve a pH of about 10.5, then the reaction continued for 16 hours. Excess 3-chloro-1,2-propanediol was scavenged with trimethylamine.

¹³C NMR spectra acquired from a Bruker 300 MHz spectrometer (samples prepared as ˜30 wt % in D₂O) confirmed the title compound: DEPT NMR (250 MHz, D2O) 51.1, (63.8, 63.9), 65.7, (65.9, 66.0, 66.1).

### Example 13

Compounds prepared substantially according to the Examples 1-12 are made and formulated into personal care compositions having otherwise conventional ingredients. The compositions are evaluated by trained panelists, with each panelist being asked to compare the inventive compositions to a conventional composition.

For hair care compositions, wet and dry feel preference and wet and dry combability is measured by asking the panelists to feel and comb two hair tresses of European virgin brown hair, commercially available from International Hair Importers and Products Inc. NY (USA), one hair tress treated with an inventive composition, the other hair tress treated with a conventional composition. Each panelist is asked to compare the tresses and state which tress is smoother to comb/feel. The answer “same” is not allowed. The reported number is the percent of panelists preferring one over the other.

For skin care compositions, panelists apply a sample (one inventive composition, one conventional composition) to a designated area on their right or left forearm. Initially, each sample is evaluated for ease of application, play time, evenness of deposit, coverage, speed of adsorbtion, shine, matte, skin moistness, heaviness, amount of grease, amount of tack, quickness of drying, overall skin feel, and overall appearance. After a designated time, each sample is again evaluated, this time for coverage, evenness of coverage, shine, matte, skin moistness, heaviness, and overall appearance.

It is understood that the present invention is not limited to the embodiments specifically disclosed and exemplified herein. Various modifications of the invention will be apparent to those skilled in the art. Such changes and modifications may be made without departing from the scope of the appended claims.

Moreover, each recited range includes all combinations and subcombinations of ranges, as well as specific numerals contained therein. Additionally, the disclosures of each patent, patent application, and publication cited or described in this document are hereby incorporated herein by reference, in their entireties.

1. A compound, including salts, of the Formula (I): 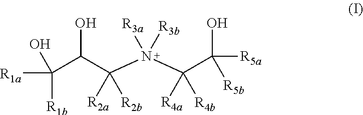
wherein:
R₁ₐ, R_{1b}, R₂ₐ, R_{2b}, R₄ₐ, R_{4b}, R₅ₐ, and R_{5b}, are, independently, H, optionally substituted C1-C6 alkyl, or R₄ₐ and R_{4b} may cooperate to form a cycloalkyl; and
R₃ₐ and R_{3b} are, independently, optionally substituted C1-C6 alkyl, with the proviso that:
when R₁ₐ, R_{1b}, R₂ₐ, R_{2b}, R₄ₐ, R_{4b}, and R₅ₐ are each H, and R₃ₐ and R_{3b} are each —CH₃, then R_{5b} is other than —CH₂OH;
when R₁ₐ, R_{1b}, R₂ₐ, R_{2b}, R₄ₐ, R_{4b}, and R₅ₐ are each H, and R₃ₐ and R_{3b} are each —CH₃, then R_{5b} is other than H;
when R₁ₐ, R_{1b}, R₂ₐ, R_{2b}, R₄ₐ, R_{4b}, R₅ₐ, and R_{5b} are each H, and R₃ₐ and R_{3b} are each —CH₂CH(OH)CH₂OH, then R_{5b} is other than —CH₂OH; and
when R₁ₐ, R_{1b}, R₂ₐ, R_{2b}, R₄ₐ, R_{4b}, R₅ₐ, and R_{5b} are each H, and R₃ₐ is —CH₃, then R_{3b} is other than —CH₂CH₂OH.

2. The compound of claim 1, wherein R₁ₐ and R_{1b} are each H.

3. The compound of claim 1, wherein R₂ₐ and R_{2b} are each H.

4. The compound of claim 1, wherein R₃ₐ and R_{3b} are each —CH₃.

5. The compound of claim 1, wherein R₃ₐ and R_{3b} are each —CH₂CH(OH)CH₂OH.

6. The compound of claim 1, wherein R₃ₐ and R_{3b} are each —CH₂CH₂OH.

7. The compound of claim 1, wherein R₃ₐ is —(CH₂)₃CH₃ and R_{3b} is —CH₂CH₂OH.

8. The compound of claim 1, wherein R₄ₐ is —CH₂OH.

9. The compound of claim 1, wherein R_{4b} is —CH₂CH₃.

10. The compound of claim 1, wherein R₄ₐ and R_{4b} cooperate to form a cyclohexyl group.

11. The compound of claim 1, having the Formula (II): 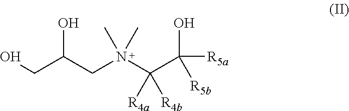
wherein:
R₄ₐ, R_{4b}, R₅ₐ, and R_{5b}, are, independently, H, optionally substituted C1-C6 alkyl, or R₄ₐ and R_{4b} may cooperate to form a cycloalkyl;
with the proviso that:
when R₄ₐ, R_{4b}, and R₅ₐ are each H, then R_{5b} is other than —CH₂OH; and
when R₄ₐ, R_{4b}, and R₅ₐ are each H, then R_{5b} is other than H.

12. A method for providing humectancy in a personal care composition, comprising including the compound of claim 1 into the personal care composition.

13. A hair care composition containing the compound of claim 1.

14. A skin care composition containing the compound of claim 1.
